(19) United States
(12) Patent Application Publication
(10) Pub. No.: US 2002/0082571 A1
(43) Pub. Date: Jun. 27, 2002

(54) Method of using lectins for agglutination and collection of menstrual flow

(76) Inventors: Howard C. Krivan, Santa Barbara, CA (US); Michael J. Oldham, Oxnard, CA (US); Richard C. Potter, Seeley Lake, MT (US)

Correspondence Address: Vorys, Sater, Seymour and Pease LLP, Suite 1111, 1828 L Street, NW, Washington, DC, 20036-5104, US

(21) Appl. No.: 10/016,327

(22) Filed: Dec. 13, 2001

Related U.S. Application Data
(63) Continuation of application No. 08/742,868, filed on Nov. 1, 1996, now abandoned.
Application No. 08/742,868 is a continuation of application No. 08/453,390, filed on May 30, 1995, now abandoned.

(51) Int. Cl. A61F 13/15; A61F 13/20; A61K 38/16

(52) U.S. Cl. 604/358; 514/8

(57) ABSTRACT

Menstrual discharge is coagulated by contacting with one or more lectins capable of agglutinating blood with the menstrual flow to coagulate the blood at least partially, thereby rendering it easier to control and collect. The lectins may be applied either alone, in neat or formulated form, or in conjunction with an intravaginal or extravaginal device. Toxic shock syndrome is prevented by administering one or more lectins capable of diminishing the infective capability of the microorganism causing toxic shock.

### BACKGROUND OF THE INVENTION

1. Field of the Invention

This invention relates generally to methods for collecting and controlling menstrual flow and, more particularly, to the use of lectins, intravaginally or extravaginally, for agglutinating and controlling menstrual flow.

2. Brief Description of the Prior Art

Women have always had to contend with the monthly inconvenience of their reproductive cycle. Various products have been introduced over the years to deal with this problem, including external absorbent pads and internal absorbent tampons of various shapes and absorbencies, as well as non-absorbent collection devices to trap menstrual discharge in liquid form.

All of these products have limitations when used, including leakage (sometimes leading to staining of clothes), odor, inconvenience, messiness, and/or the possibility of infection. These problems stem from the fact that menstrual discharge consists primarily of uncoagulated blood and, hence, is a low-viscosity fluid which flows readily (thereby contributing to leakage through or around a device), has high potential for staining (particularly for staining clothes if device leakage occurs and, also, the fingers and hands upon device insertion or removal), and is readily capable of harboring and nurturing microorganisms which are capable of propagating strong odors and even infection, such as toxic shock syndrome (TSS). As a result, it is evident that a purely mechanical device such as a pad, tampon or non-absorbent collector is limited in its effectiveness because of the fluid properties inherent in uncoagulated blood.

Accordingly, a need has existed for a method to either supplant or augment purely mechanical means for control of menstrual flow.

### SUMMARY OF THE INVENTION

The need for an improved method of collection and control of menstrual discharge has now been alleviated by the method of this invention, according to which one or more lectins capable of agglutinating blood are contacted with the menstrual blood to coagulate the blood at least partially, thereby rendering it easier to control and collect. The lectins may be applied either alone, in neat or formulated form, or in conjunction with an intravaginal or extravaginal device.

Accordingly, it is an object of the invention to provide an improved method for collection and control of menstrual discharge in humans and other menstruating animals.

It is a further object to agglutinate menstrual discharge intravaginally.

It is a further object to agglutinate menstrual discharge extravaginally.

It is a further object to provide a method of prophylaxis against TSS.

Other objects of the invention will become apparent from the following detailed description.

### DETAILED DESCRIPTION OF THE INVENTION AND PREFERRED EMBODIMENTS

Lectins are carbohydrate-binding proteins of non-immune origin that agglutinate (coagulate) cells or precipitate polysaccharides or glycoconjugates (proteins or lipids conjugated to oligo- or polysaccharides). They are widely distributed and have been isolated from both plant and animal sources. Their reactions with living cells are based on their ability to bind with antibody-like specificity to particular arrangements of the sugar residues that make up oligo- or polysaccharides.

Glycoconjugates are abundant on the surface of cell membranes in multicellular animals (including mammals) as well as on the surface of single-celled organisms. Similarly, the cell walls and capsules of bacteria and the envelopes of viruses contain structural polysaccharides and/or glycoproteins. The carbohydrate moieties of these molecules which are displayed on the cell surface exhibit great variety in structure and composition that serves to distinguish the types of cells and to serve as a signal to other cells or materials which come into contact with the cell. When lectins recognize and bind to certain carbohydrate moieties, they may serve to cross-link and agglutinate the cells bearing the binding groups (hence leading to the alternate name of agglutinins for lectins).

Lectins are particularly noteworthy for their ability to agglutinate erythrocytes (red blood cells). Indeed, this phenomenon led to the original discovery of lectins. Moreover, certain lectins can selectively bind with specific variations of the carbohydrate moieties of glycoproteins in the erythrocyte plasma membrane. This serves as the basis for modern blood typing techniques.

Glycoconjugates found on the surface of both leukocytes (white blood cells) and tissue from the uterine lining can also bind with lectins.

A representative listing of lectins, the abbreviations by which they are referred to, and their sources are given in Table 1.

TABLE 1
Representative Lectins, Abbreviations & Sources

| | |
|---|---|
| AAP | Aaptos papillata (sponge) |
| AAnA | Anguilla anguilla (eel serum) |
| AAurA | Aleuria aurantia (orange peel fungus) |
| ABA | Agaricus bisporus (common mushroom) |
| ABrA | Amphicarpaea bracteata (hog-peanut) |
| AGT | Agardhiella tenera (red alga) |
| AL | Hippeastrum hybrid (amaryllis bulb) |
| APA | Abrus precatorius (Jequirty bean) |
| AS | Avena sativa (oat) |
| BDA | Bryonia dioica (white bryony) |
| BPA | Bauhinia purpurea alba (camel s foot tree) |
| CA | Colchicum autumnale (meadow saffron) |
| CAA | Caragana arborescens (Siberian pea tree) |
| CCA | Cancer antennarius (California or blue crab) |
| ConA | Canavalia ensiformis (jack bean) |
| CPA | Cicer arietinum (chickpea) |
| CSA | Cytisus scoparius (Scotch broom) |
| DBA | Dolichos biflorus (horse gram) |
| DSA | Datura stramonium (jimsonweed, thorn apple) |
| ECA | Erythrina cristagalli (coral tree) |
| ECorA | Erythrina corrallodendrin (coral tree) |
| EEA | Evonymus europaeus (spindle tree) |
| GNA | Galanthus nivalis (snowdrop bulb) |
| GSA-I/GSA-II | Griffonia simplicifolia (African legume) |
| HAA | Helix aspersa (garden snail) |
| HPA | Helix pomatia (Roman or edible snail) |
| JAC (Jacalin) | Atocarpus integrifolia (jackfruit) |
| LAA | Laburnum alpinum (golden chain) |
| LBA | Phaseolus lunatus (also limensis) (lima bean) |
| LCA (LcH) | Lens culinaris (lentil) |
| LEA | Lycopersicon esculentum (tomato) |
| LFA | Limax flavus (garden slug) |
| LIP (Limulin) | Limulus polyphemus (horseshoe crab) |
| LOA | Lathyrus odoratus (sweet pea) |
| LTA (LOTUS) | Lotus tetragonolobus (asparagus pea) |
| MAA | Maackla amurensis (maackla) |
| MIH | Mangifera indica (mango) |
| MPA | Maclura pomifera (Osage orange) |
| NPL (NPA) | Narcissus pseudonarcissus (daffodil) |
| PAA | Persea americana (avocado) |
| PHA (PHA-L) | Phaseolus vulgaris (red kidney bean) |
| PNA | Arachis hypogaea (peanut) |
| PSA | Pisum sativum (pea) |
| PTP | Ptilota plumosa (red alga) |
| PWA | Phytolacca americana (pokeweed) |
| PTAgalactose | Psophocarpus tetragonolobus (winged bean) |
| PTAgalNac | Psophocarpus tetragonolobus (winged bean) |
| RCA-I/RCA-II | Ricinus communis (castor bean) |
| RPA | Robinia pseudoacacia (black locust) |
| SBA | Glycine max (soybean) |
| SJA | Sophora japonica (Japanese pagoda tree) |
| SNA | Sambucus nigra (elderberry) |
| SOF | Soduim fragile (green alga) |
| STA | Solanum tuberosum (potato) |
| TKA | Trichosanthes kirilowii (China gourd) |
| TL | Tulipa gesneriana (tulip) |
| TMT | Tomentine (seaweed Codium tomentosum) |
| UEA-I/UEA-II | Ulex europaeus (gorse or furze seeds) |
| VAA | Viscum album (European mistletoe) |
| VFA | Vicia faba (fava bean) |
| VGA | Vicia graminea (herb) |
| VRA | Vigna radiata (mung bean) |
| VSA | Vicia sativa (vetch) |
| VVA | Vicia villosa (hairy vetch) |
| WFA | Wisteria floribunda (Japanese wisteria) |
| WGA | Triticum vulgaris (wheat germ) |
| suc-WGA | Succinylated WGA |

While the lectins listed above are representative of useful lectins according to the invention, it is to be understood that other lectins may be discovered which are also useful for these purposes.

According to the invention, one or more lectins are administered during the course of menses as a means of agglutinating the constituents of the menstrual flow such as the erythrocytes, leukocytes, and uterine tissue fragments which comprise the menstrual discharge. Such agglutination will serve to immobilize at least the components of the menstrual discharge that are most prone to staining.

Many lectins are capable of agglutinating blood and are, therefore, useful for the invention. ConA, WGA, and LCA are examples of lectins capable of agglutinating all types of human blood. In general, those lectins which bind more strongly to menstrual discharge than to vaginal epithelial surfaces are preferred.

In the simplest embodiment of the invention, intravaginal administration of lectins can be effected in neat form, in aqueous solution, or in formulated form, wherein the lectin is dissolved or dispersed in an excipient such as an ointment, cream, jelly, foam or the like. The formulations can also incorporate adjuvants such as thickeners and/or lubricants. Such formulations can be delivered manually or with the aid of devices such as a pump, aerosol, or applicator. Delivery from such devices can often be facilitated by incorporating a long, tubelike applicator capable of insertion into the vagina. As a sanitary precaution, such applicators can be disposable after a single use.

Other, similarly efficacious methods of intravaginal administration of neat, dissolved or formulated lectins will be readily apparent to one of ordinary skill in the art. In this embodiment of the invention, menstrual fluid emerging from the cervical os comes into contact with lectins which have been administered intravaginally. Agglutination occurs shortly thereafter, causing the formation of a gelatinized mass and thereby immobilizing the menstrual fluid and preventing leakage from the vagina. This mass can subsequently be removed from the vagina manually or by use of mechanical methods such as a mechanical probe or douche. Alternatively, the mass can be expelled by the movement of the vaginal walls as a result of normal, everyday activity.

Another embodiment of the invention entails the application of lectins extravaginally, on or around the female external genitalia, in neat form, in solution, or incorporated into a vehicle. In this case, menstrual discharge is agglutinated upon exiting from the vagina. The agglutinated mass is then readily removed from the area of the external genitalia or from undergarments.

In a more preferred embodiment of the invention, a lectin or mixture of lectins can be administered intravaginally by prior incorporation of the lectins into a mechanical device. Such a device can be inherently absorbent or non-absorbent or can contain a combination of absorbent and non-absorbent surfaces. Examples of non-absorbent devices include: a solid or hollow (tubular), flexible rod with a circular, oval, pleated or rectangular cross-sectional shape; a solid or hollow (tubular), flexible ring with a circular, oval, or rectangular cross-sectional shape; a cup-shaped device, such as a diaphragm; or any of a variety of non-absorbent collection devices previously disclosed in the art. Examples of such devices are disclosed in Contente et al, U.S. Pat. No. 5,295,984; Zöller, U.S. Pat. No. 3,845,766; Zalucki, U.S. Pat. No. 3,841,333; and Waldron, U.S. Pat. No. 3,626,942, and U.S. Pat. No. 3,404,682. Such devices are typically fabricated from flexible polymers, such as synthetic thermoplastics or natural rubbers. An example of an absorbent device suitable for the invention is a conventional tampon, which is commonly fabricated from natural or synthetic fibers. Tampons are especially preferred devices for this invention. Other intravaginal absorbent devices have been disclosed in the art, e.g., in Davis, U.S. Pat. No. 3,983,874; Steiger, U.S. Pat. No. 3,216,422; and Nolan, U.S. Pat. No. 3,128,767.

Lectins can be incorporated into any of these devices by a variety of means. Such means include spraying a lectin solution onto the surface of the device and allowing a film to form as a result of dissipating the solvent. Another means is to dip the device into a solution of lectins and allowing the lectins to subsequently dry into a film. Still another means is to spray or dip the device with a lectin formulation containing adjuvants, such as lubricants or thickeners. Alternatively, lectins in neat or dissolved form, or lectins in combination with various adjuvants, can be milled or otherwise dispersed into the device during the fabrication of the device such that all or a portion of the lectins are exposed superficially on the device. Devices with a hollow reservoir and permeable walls can be charged with a lectin-containing fluid. Various modifications of devices can be effected in order to increase the surface area of exposed lectins and thereby increase the efficiency of the invention, such as constructing the device from a porous material or incorporating appendages having a high surface area to volume ratio, such as small spheroids.

Other devices and variations thereof for the intravaginal administration of lectins useful for agglutination of menstrual fluid will be apparent to one of ordinary skill in the art. In this embodiment of the invention, menstrual fluid emerging from the cervical os comes into contact with lectin molecules on the external or internal surfaces of the intravaginal device. Agglutination occurs shortly thereafter, causing immobilization of the menstrual fluid on the surface of the device, thus preventing leakage from the vagina. The device can subsequently be conveniently removed from the vagina manually or with a mechanical probe.

In another preferred embodiment, lectins are used extravaginally by prior incorporation of the lectins into a mechanical device. Such a device can be inherently absorbent or non-absorbent or can contain a combination of absorbent and non-absorbent surfaces. However, most typically the device is of a predominantly absorbent type, such as a pad, napkin, panty, or panty-liner. Such devices are commonly fabricated from natural or synthetic fibers, either woven or non-woven, and are generally designed to conform with the female external genitalia. Lectins can be incorporated into any of these devices by a variety of means. Such means include spraying a lectin solution onto the surface of the device and allowing a film to form as a result of dissipating the solvent. Another means is to dip the absorbent device into a solution of lectins, thereby saturating and impregnating the device with the solution, and then allowing the lectins to dry into a coating. Still another means is to spray or dip the device with a lectin formulation containing adjuvants, such as moisturizers or thickeners. Alternatively, lectins in neat or dissolved form, or lectins in combination with various adjuvants, can be milled or otherwise dispersed into the device during the fabrication of the device such that all or a portion of the lectins are exposed on both the external and internal surfaces of the device. A non-absorbent extravaginal device suitable for the method of this invention is a solid shield designed to conform to the external female genitalia, preferably constructed of a flexible polymeric material. The interior surface of the shield can be coated with lectins by utilizing any of the aforesaid techniques.

In this embodiment of the invention, menstrual fluid emerging from the vaginal opening comes into contact with lectin molecules on the external or internal surfaces of the extravaginal device. Agglutination occurs shortly thereafter, causing immobilization of the menstrual fluid on the surface of the device. Other variations and modifications will be apparent to those of ordinary skill in the art.

It is evident that the methods of this invention greatly reduce the chance for menstrual blood to escape the vagina and thereby cause unwanted leakage, staining of clothes, anxiety, and discomfort for the menstruator.

Since menstrual discharge frequently harbors microorganisms which can lead to the development of foul odors or which can lead to various infections, it is evident that another advantage of the invention is to ameliorate such events because microorganisms grow and multiply much less readily in a hardened medium, such as agglutinated blood. This relates to another embodiment of the invention, wherein pathogens which lead to the development of TSS can be neutralized by the inclusion of lectins with binding specificities for the pathogens. Staphylococcus aureus has been implicated as the primary pathogen for TSS and, in an especially preferred embodiment of the invention, a lectin or-lectins which bind to S. aureus are impregnated into or coated onto absorbent intravaginal devices, such as tampons. Such lectins can be added in addition to, or instead of, blood-coagulating lectins and can also be used in conjunction with extravaginal absorbent devices.

A variety of lectins can be used for this purpose, alone or in combination. For example, S. aureus can be bound by the lectins WGA (Davidson, S K et al, J Clin Microbiol 15: 547-53 (1982)), ConA (Reeder, N J et al, J Immunol 196: 334-40 (1971)), and LIP (Gilbride K J et al, Prog Clin Biol Res 29: 525-35 (1979)). WGA and ConA have a binding affinity for N-acetyl-D-glucosamine residues expressed on a bacterial surface (Doyle, R J, Lectin-Microorganism Interactions, Marcel-Dekker (New York), 43-55 (1994)), and strains of S. aureus are known to express such residues (Slifkin, M, Lectin-Microorganism Interactions, Marcel-Dekker (New York), 144-5 (1994)).

Yet another advantage of the invention is that, because of the more efficient immobilization of menstrual matter afforded by lectins, conventional sanitary pads and tampons can be re-designed, incorporating lectins, into more compact and, hence, more comfortable and less noticeable product forms.

It is intended that each embodiment of this invention shall be applicable to both human females and other female mammals capable of menstruation, such as female cats or female dogs. Owners of such cats and dogs, who utilize absorbent napkins to collect the menses of their animals, will be able to use the methods of this invention for the avoidance of leakage, attendant staining of carpeting and furnishings, and reduction of odor.

### EXAMPLE

### Agglutination of Human Blood Ex Vivo

Highly purified, essentially salt-free lectin from Triticum vulgaris (WGA, wheat germ agglutinin) was tested for its ability to agglutinate human erythrocytes. As this lectin is specific for cells that contain N-acetylglucosamine and/or sialic acid carbohydrate residues, which are present on A, B, O and AB type erythrocytes, it is defined as a non-specific lectin for these red cell types. Erythrocytes were washed three times in 10 volumes of 0.01 M sodium phosphate, pH 7.2, containing 0.15 M sodium chloride (PBS) and diluted to a 2.5% suspension. Two-fold serial dilutions of 1 mg/mL lectin (50 μL) were performed with PBS in V-bottom or U-bottom microtiter plates. Then, 50 μL of the freshly prepared 2.5% erythrocyte suspension was added to each well. The plates were gently tapped, and the erythrocytes were allowed to settle at room temperature (ca. 22° C.). WGA was found to agglutinate the human erythrocytes at concentrations ranging from 10 to 20 μg lectin/mL after one hour incubation at room temperature.

Unwashed, whole blood (50 μL) mixed 1:1 with a 50 μL solution of WGA (1 mg lectin/mL) will also agglutinate erythrocytes within 5 to 10 seconds. Other mammalian erythrocytes, such as sheep and rabbit, are agglutinated by WGA in a similar fashion and at approximately the same concentration range of lectin.

The invention having now been described, it should be understood that it may be embodied in other specific forms or variations without departing from its spirit or essential characteristics. Accordingly, the embodiments described above are to be considered in all respects as illustrative and not restrictive, the scope of the invention being indicated by the appended claims rather than by the foregoing description, and all changes which come within the meaning and range of equivalency of the claims are intended to be embraced therein.

We claim:

1. A method of coagulating menstrual flow in humans and other animals comprising contacting menstrual flow with at least one lectin capable of agglutinating at least one of the constituents of said menstrual flow.

2. The method of claim 1 wherein said lectin is capable of agglutinating erythrocytes, leukocytes, and/or tissue fragments comprising said menstrual flow.

3. The method of claim 1 wherein said lectin is selected from the group consisting of WGA, SBA, ConA, and LCA.

4. The method of claim 1 wherein said lectin is administered intravaginally.

5. The method of claim 4 wherein said lectin is in neat form.

6. The method of claim 4 wherein said lectin is contained in a vehicle comprising excipients or adjuvants.

7. The method of claim 4 wherein said lectins are contained in an absorbent device.

8. The method of claim 4 wherein said lectins are contained in a non-absorbent device.

9. The method of claim 1 wherein said lectins are applied extravaginally.

10. The method of claim 9 wherein said lectins are in neat form.

11. The method of claim 9 wherein said lectins are contained in a vehicle comprising excipients or adjuvants.

12. The method of claim 9 wherein said lectins are contained in an absorbent device.

13. The method of claim 9 wherein said lectins are contained in a non-absorbent device.

14. A method of preventing toxic shock syndrome in humans caused by pathogenic microorganisms which comprises administering to a human in danger of infection with a pathogenic microorganism causing toxic shock syndrome at least one lectin capable of diminishing the infective capability of said microorganism in an amount effective to diminish the infective capability of said microorganism.

15. The method of claim 14 wherein said microorganism is a strain of Staphylococcus aureus.

16. The method of claim 14 wherein said lectins are selected from the group consisting of WGA, SBA, ConA, and LIP.

17. The method of claim 14 wherein said lectin is administered intravaginally.

18. The method of claim 14 wherein said lectin is in neat form.

19. The method of claim 14 wherein said lectin is contained in a vehicle comprising excipients or adjuvants.

20. The method of claim 14 wherein said lectin is incorporated in an absorbent device.

21. The method of claim 20 wherein said absorbent device is a tampon.

22. The method of claim 20 wherein said absorbent device is applied extravaginally.
